(19) United States
(12) Patent Application Publication
(10) Pub. No.: US 2009/0274734 A1
(43) Pub. Date: Nov. 5, 2009

(54) METHOD FOR OBTAINING HOLLOW PARTICLES

(75) Inventors: Wilhelmina Francisca Daamen, Afferden (NL); Petrus Johannes Geutjes, Nijmegen (NL); Antonius Henricus Minardus Severus Maria Van Kuppevelt, Nijmegen (NL)

Correspondence Address: BROWDY AND NEIMARK, P.L.L.C.;624 NINTH STREET, NW, SUITE 300, WASHINGTON, DC, 20001-5303, US

(73) Assignee: Stichting Katholieke Universiteit, Nijmegen (NL)

(21) Appl. No.: 12/095,917

(22) PCT Filed: Dec. 2, 2005
(86) PCT No.: PCT/NL05/00828

§ 371 Date: Oct. 28, 2008

(51) Int. Cl. A61K 9/14; B29C 35/16; B32B 9/04; A61K 39/395; A61K 38/43; A61K 8/02; A61P 17/00

(52) U.S. Cl. 424/401; 264/28; 428/402.2; 424/130.1; 424/489; 424/9.1; 424/94.1

(57) ABSTRACT

Described is a method for obtaining hollow particles, having a particle wall and a particle lumen, the particle having dimensions of between 1 nm and 100 μm, from a mixture comprising a liquid medium comprising at least one colloid or solute, the method comprising freezing said mixture and lyophilising the obtained frozen mixture, characterised in that a volume of at least 0.1 μl of the mixture is subjected to a freezing step comprising: (a) (1) quench freezing the mixture resulting in a quench frozen mixture, and (2) incubating said quench frozen mixture at a temperature above the quench freezing temperature and below the melting point of the liquid medium, orb) (1) reducing the temperature of the mixture at a rate of 1 to 100° C./minute to below the freezing temperature of the mixture and (2) incubating said frozen mixture at a temperature above the temperature of the mixture and below the melting point of the liquid medium. Further, hollow particles obtainable by the said method, compositions comprising said hollow particles and uses thereof are described.

The present invention relates to a method for obtaining hollow particles, hollow particles obtainable by the said method, compositions comprising said hollow particles and uses thereof.

Different methods for the preparation of hollow particles in the nanometer or micrometer range are known in the art, herein also referred to as “small hollow particles”.

Such small hollow particles are of interest in a growing variety of medical, pharmaceutical, biomedical, cosmetic, diagnostic, chemical and other applications.

Examples of small hollow particles are so-called liposomes prepared from lipids and/or other amphipathic molecules, described by Bangan (reviewed in Bangan, A. D. et al. Bioassays. 1995 Dec; 17(12):1081-1088). Normally liposomes consist of a spherical lipid bilayer enclosing an inner compartment or a hollow core. Results with liposomes with respect to the above applications are limited, possibly due to e.g. mechanical instability, and liposomes have only limited application.

Hollow particles from chitosan have also been described in the art. E.g. U.S. Pat. No. 6,238,705 describes particles having a material of e.g. alginate, coated with chitosan. The core can be removed resulting in a hollow chitosan particle. A major disadvantage of these particles is the high solubility in acid and the tendency to lose integrity.

Hollow particles have also been described prepared from specially engineered peptides with self-assembling properties, e.g. amphiphiles containing both soluble and insoluble domains, comparable to lipids. It has for example been reported that spherical assemblies can be prepared from diblock copolypeptides that self-assemble (Belomo et al; Nature Materials 2004; 3:244-248).

The possibility of formation of hollow particles as described above depends strongly, if not entirely, on the properties of compounds. For example, formation of liposomes or engineered hollow peptide particles is a self-assembly process that is driven by e.g. the amphipathic nature of the compounds, whereas chitosan particle formation depends on the binding of chitosan with materials with cross-linking properties. Most compounds, like natural proteins, lack these strict properties and can not successfully be applied (i.e. they are unsuitable for preparing hollow particles) in the methods for the formation of hollow particles known in the art.

Further, US patent application US 2005/017802 describes a method and apparatus for producing particles from solutes like peptides, proteins, sugars and polymers. The method comprises the steps of providing a solution with a solute, mixing said solution with a compressed fluid and flowing the mixture across a pressure drop into an expansion chamber, wherein the mixture is atomised into individual particles with a diameter of 0.01 micrometer to about 200 micrometer. As the compressed fluid expands and decompresses, the temperature is reduced below the freezing point of the atomised particles. The individual atomised particles are subsequently freeze-dried to evaporate the solvent, forming solid particles having a size substantially equal to the atomised particles.

In addition to the fact that a complex apparatus is needed, the particles obtained have the morphology and size of the atomized individual particles.

U.S. Pat. No. 6,284,282 discloses a method for preparation of a dry powder of a therapeutic protein suitable for administration via pulmonary delivery. This method requires atomising a liquid formulation into individual particles having an average diameter of about 5 to about 30 micrometer, followed by freezing and freeze-drying of said droplets and subsequent drying. The particles obtained are however solid spherical particles.

Both the above methods of US patent application US 2005/017802 and U.S. Pat. No. 6,284,282 do not allow for further, easy and convenient manipulation of properties of the particles, e.g. size, morphology or distribution of any compound of interest throughout the particle, as these properties are already dictated upon atomisation into individual particles, resulting in particles, without the possibility to further manipulate the formation of the particle.

There is thus need for an easy, versatile, convenient and general applicable method for obtaining small particles, in particular hollow particles, allowing the preparation of small hollow particles from a wide variety of materials, not necessarily limited to engineered compounds or amphipathic materials, but also e.g. solutes, colloids, dispersions, compounds in suspension and others, without the need of engineering said materials, and wherein properties of said particles, like e.g. size, morphology composition and others, can easily be manipulated, thus providing particles tailored to specific needs.

### DETAILED DESCRIPTION OF THE INVENTION

It is the aim of the present invention to solve one or more of the above-mentioned problems and/or disadvantages in the preparation of small hollow particles.

It is now surprisingly found that one or more of the above-mentioned problems can be solved by providing a method for the preparation of hollow particles having a particle wall and a particle lumen, the particle having dimensions of between 1 nm and 100 μm, from a mixture comprising a liquid medium comprising at least one colloid or solute, the method comprising freezing said mixture and lyophilising the obtained frozen mixture, characterised in that a volume of at least 0.1 μl of the mixture is subjected to a freezing step comprising:

(a)(1) quench freezing the mixture resulting in a quench frozen mixture, and (2) incubating said quench frozen mixture at a temperature above the quench freezing temperature and below the melting point of the liquid medium, or

(b)(1) reducing the temperature of the mixture at a rate of 1 to 100 ° C./minute to below the freezing temperature of the mixture, and (2) incubating said frozen mixture at a temperature above the glasstemperature of the mixture and below the melting point of the liquid medium.

Herein, the term “particle” includes any structure that is an aggregation of sufficiently many molecules that it can be assigned properties such as volume and/or density. As outlined above, “the dimensions of which are between 1 nm and 100 μm” means to refer to such structures having a maximal length, width or diameter from 1 nm to 100 micrometer, i.e. in the nanometer and micrometer range. Preferably the particles have a maximal length, width or diameter in the range of 20 nm to 60 μm. According to the method hollow particles can be provided i.e. particles comprising an outer wall (also referred to as “wall”), i.e. a wall that is in contact with the surrounding environment and encloses an inner lumen. The inner lumen. (also referred to as “lumen”) of such hollow particle can accommodate e.g. a liquid, or gas, in which other materials like drugs or vitamins or magnetic particles can be dissolved or dispersed. It can also accommodate a solid compound, even at such a high concentrations that the particle can be considered to be massive, with a wall and a lumen loaded with a solid compound. In another embodiment the method provides for a hollow particle that is porous, i.e. having small pores throughout the particle wall, thereby modifying the density in comparison to a non-porous hollow particle formed from the same material. In case of a hollow particle, such pores (or small channels) can connect the inner core with the surrounding environment.

In the method according to the invention at least one colloid or solute is mixed with a liquid medium. The term colloid is known in the art and includes any substance which is dispersed in such a fine state or sub-division in a medium that it does not settle out in the liquid medium, but not in so fine a state of sub-division that it can be said to be truly dissolved, and is herein also referred to as “particle material” or “material for the preparation of particles”.

With a solute any substance that can be dissolved in fluid is meant. The dissolved substance is defined as the solute and the dissolving fluid is called the solvent, which together form a solution.

The term “liquid medium” is known in the art and is here directed to any suitable liquid that is used as carrier for e.g. the solute or colloid used. In general, the liquid medium, within the context of the current invention, comprises more than 50% of the total volume of the mixture, i.e. forms the bulk of the mixture. The liquid medium can be any suitable medium, like water, preferably comprising a suitable buffer: Preferably the medium is chosen in that e.g. during the later lyophilisation step, the bulk of the liquid medium can easily be removed, e.g. by sublimation, resulting in substantially dry hollow particles. Preferably more than 90%, more preferably more than 95% and most preferably more than 98% of the liquid medium is removed.

The volume of the mixture that is subjected to the freezing step is at least 0.1 microliter (μl), and can for example be between 0.1 microliter and 10 ml, in order to obtain hollow particles of the envisaged size. It has surprisingly been found that within a mixture-volume of at least 0.1 microliter, e.g. in the form of a droplet or a thin layer of the mixture on a metal plate, a plurality of small particles within the nanometer and micrometer range are formed, which in addition surprisingly allows for effective manipulations and handling of the properties, such as size, morphology and composition, of the numerous particles, thus providing a versatile method for formation of a wide variety of particles.

In contrast, the methods comprising atomisation prior to freezing, as known in the art and as discussed above, leads to formation of frozen droplets with a much smaller volume which already have approximately the size of an individual particle, e.g. have an average diameter of about 5 to about 30 micrometer. The size of atomised droplets does not allow for the formation on numerous particles within the droplet, but represents an individual particle and does not allow for further efficient manipulation of the properties of the individual particle.

It has been found that according to the invention the freezing step can either comprise (a) (1) quench freezing the mixture and incubating said quench frozen droplets at a temperature above the quench freezing temperature and (2) below the melting point of the liquid medium, or

(b)(1) reducing the temperature of the mixture at a rate of 1 to 100° C./minute to below the freezing temperature of the mixture, and (2) incubating said frozen mixture at a temperature above the glasstemperature of the mixture and below the melting point of the liquid medium.

Herein “quench freezing” refers to very rapid freezing of the material, so that the mixture is totally frozen preferably within 80 seconds, preferably 30 seconds, more preferably 20 seconds after subjecting the mixture, e.g. droplets thereof, to freezing.

After quench freezing the mixture, said quench frozen material is incubated at a temperature above the quench freezing temperature and below the melting point of the liquid medium. It was found that by the combination of quench freezing and further incubating at a temperature above the quench freezing temperature and below the melting point of the liquid medium allows for the formation of the particles. Incubation at a temperature above the quench freezing temperature and below the melting point of the liquid medium can be performed after the quench frozen mixture, e.g. quench frozen droplets, has been formed, e.g. by placing the frozen droplets in another medium, or by increasing the temperature of the freezing medium.

Preferably the incubation temperature is below the melting temperature of the liquid medium, but above the glass-temperature (i.e. the temperature below which the molecules in the mixture have very little mobility; glass temperature characterises the transition from true solid to viscous liquid (usually in non-crystalline solids which do not have a sharp melting point)) of the mixture. Methods for determination of the glass-temperature are known to the person skilled in the art and within the context of the current invention is preferably performed by differential scanning calorimetry (DSC) using a SCC5200 (SEIKO Instruments).

The freezing step can also be performed by reducing the temperature of the mixture at a rate of 1° C. to 100° C./minute, preferably 3° C. to 75° C./minute, even more preferably 5° C. to 40° C./minute and incubating said frozen mixture at a temperature above the glasstemperature of the mixture and below the melting point of the liquid medium. It has been found that, in particular with, but not limited to, higher volumes of 100 μl or more, preferably 1 ml or more, of the mixture (e.g. 4 ml), within the context of the current invention, freezing the mixture by reducing the temperature of the mixture at a rate as mentioned above, surprisingly allows for the hollow particle formation according to the invention. The person skilled in the art understands that reducing the temperature can be a continuous process at a constant rate (e.g. 10° C./min), but can likewise be a continues process at an increasing or decreasing rate (e.g. from 1° C./min to 5° C./min), or be e.g. a non-continuous process at either a constant or changing rate (e.g. 2 minutes at a rate of 20° C./minute, followed by 3 minutes at a rate of e.g. 0° C./minute or 5° C./minute). Preferably the mixture is frozen by reducing the temperature within a time period of 30 seconds to 60 minutes. The freezing step (b) comprises incubating the obtained frozen mixture at a temperature above the glass-temperature of the mixture. As known by the person skilled in the art, the glasstemperature is, amongst others, depending on the composition of the mixture, the glasstemperature can for example be a temperature above −120° C.

After the freezing step, the frozen mixture is lyophilised. The term “lyophilisation” or “lyophilised” or “freeze drying” is known in the art and encompasses dehydration or sublimation by freezing and reducing the pressure to allow a frozen solvent in the material to sublimate directly from the solid phase to gas. Various methods and apparatuses which can be used are known in the art (see Skrabanja, A. T. P. et al. PDA J Pharm Sci Technol. 1994 November-December; 48(6):311-317).

During lyophilisation, conditions are chosen as such that the liquid medium will evaporate/sublime, whereas the solute and/or colloid used will not or essentially not be removed. The person skilled in the art understands or can, within the context of the current invention, easily learn by straightforward experimentation to select suitable parameters such as pressure, temperature, time and others. After lyophilisation, the solute and/or colloid are comprised in the particle wall and constitute the particle material, optionally in combination with other materials present in the particle, such as drugs, biomolecules, or contrast agents.

It has been found that quench freezing of step (a) is preferably performed by using small droplets. Therefore according to a preferred embodiment the volume of the droplets of step (a) is between 0.1 μl and 1000 μl, preferably between 1 μl and 100 μl, more preferably between 2 μl and 50 μl, even more preferably between 3 μl and 30 μl, most preferably between 5 μl and 25 μl.

Droplets of the above mentioned volumes provides upon quench freezing frozen droplets which each comprise a plurality of particles that can be suitably manipulated, and provide good and high yield of particles according to the invention. As will be understood by the person skilled in the art, depending on the volume of the droplets, the time to freeze the droplet will vary. The skilled person will understand that the volume of the particles can suitably be chosen, e.g. depending on the volume of the particle material or the required size. In connection therewith, and as will be exemplified in the methods, size and morphology of the particles can be advantageously. adjusted/modified to particular needs or requirements, e.g. in forming a hollow particle of required size.

The volume of a droplet to be quench frozen can be chosen by using methods known in the art, and can for example involve calibrating so-called micropipettes.

According to a further preferred embodiment of the invention, the quench freezing step (a) comprises freezing the mixture by contacting with a freezing medium, the freezing medium having a temperature of below the freezing temperature of the mixture.

Upon immersing e.g. a droplet of the mixture comprising a liquid medium and at least one colloid or solute in a freezing medium a frozen droplet can very quickly be formed, and immersion in or on a freezing medium thus can allow for a versatile manner for quench freezing the mixture. The freezing medium can be any material, including any liquid, gas or solid, as long as the freezing medium has a temperature, or can be brought to a temperature, preferably at atmospheric pressure, that is below the freezing temperature of the mixture comprising the liquid medium and at least one colloid or solute, in order to form a frozen mixture, e.g. a frozen droplet by quench freezing.

According to another embodiment the freezing medium in which the droplet is immersed has a temperature between −270° C. and +20° C., preferably between −230° C. and −50° C. It is found that the rate of the freezing process, as well as the incubation time, appear an important variable in obtaining the required morphology of the hollow particles. Depending on the colloid or solute used, it has generally been found that when the freezing rate is slowed (e.g. by a higher temperature of the freezing medium or a higher volume of the droplet, or by a different freezing medium) less globular particles are obtained and more sheet-like structures are found. In addition it has in general been found that when freezing rate is increased, e.g. by reducing the volume of the droplet or by choosing a freezing medium having a lower temperature, a tendency for the formation of globular structures (particles) is observed. The person skilled in the art will by straightforward experimentation according to the teachings herein easily be capable of learning the suitable conditions of the freezing medium for obtaining the required particle.

According to a preferred embodiment of the invention the freezing medium comprises liquid nitrogen. It has been found that liquid nitrogen is suitably used for efficient (quench) freezing and the formation of numerous particles within the quench frozen mixture, e.g. when an organic or inorganic liquid medium is applied.

Other freezing media, like cryogenic liquids, CF4, CH4, propane, helium, and others generally known it the art, e.g. ethanol/CO₂ or methanol/CO₂ can also be successfully applied as long as the freezing medium has a temperature of below the freezing temperature of the mixture comprising a liquid medium and at least one solute or colloid, in order to form a frozen mixture, e.g. a frozen droplet of the mixture, thus allowing for the formation of numerous particles within said droplet. The person skilled in the art will, by straightforward experimentation, be capable of determining a suitable freezing media for obtaining the required particle, by comparison of e.g. freezing media with different temperatures.

According to another embodiment of the current invention the incubation of the quench frozen mixture is carried out at a temperature between −200° C. and 0° C., preferably between −140° C. and 0° C., most preferably between −20° C. and 0° C. It was found that adjusting the incubation temperature can be suitably applied to adjust the size of the envisaged particle. For example, with a lower temperature smaller particles, can be obtained after lyophilisation (e.g. −80° C. for the protein elastin), in comparison with a higher temperature. It is the inventors belief that some micro-molecular motion occurred during the procedure, thus influencing the size of the particle obtained. Also it is to be contemplated that for other. particle materials, the above can be the other way around, e.g. that higher temperature leads to formation of smaller particles. A skilled person in the art will, by the teaching disclosed herein, easily be able to adjust the temperature to obtain particles with the envisaged properties, e.g. by comparing particles obtained at different incubation temperatures.

It has been found that freezing of step (b) by reducing the temperature of the mixture at a rate of 1° C. to 100° C./minute can be advantageously used, but is not limited to, higher volumes of the mixture. It is therefore another embodiment of the current invention that the volume of the mixture of step (b) is between 0.1 ml and 100 ml, preferably between 0.5 ml and 50 ml, most preferably between 1.0 ml and 10 ml.

Volumes of the mixture of the above mentioned volumes provides upon freezing according to step (b), a frozen mixture that comprises a plurality of particles that can be suitably manipulated, and provide good and high yields of particles according to the invention. As will be understood by the person skilled in the art, depending on the volume of the mixture, the time to freeze the mixture will vary (e.g. at a given freezing temperature), but normally occurs within 30 seconds to 60 minutes. The skilled person will understand that the volume of the particles can suitably be chosen, e.g. depending on the volume of the particle material or the envisaged size of the particle. In connection therewith, and as will be exemplified in the methods, size and morphology of the particles can be advantageously adjusted/modified to particular needs or requirements, e.g. for forming a hollow particle of required size.

The volume of a droplet to be frozen can be chosen by using methods known in the art, and can for example involve calibrating so-called micropipettes.

According to a further embodiment of the current invention, the lyophilising step (c) comprises the steps of
(c1) applying a temperature which is below the freezing temperature. of the liquid medium, at a pressure between 0-1000 Pascal (Pa), preferably 20-500 Pa, most preferably 50-200 Pa,: for 1 hour to 7 days, more preferably 2-24 hours, most preferably 4-18 hours; followed by
(c2) increasing the temperature to between −120° C and +40° C. over a period of 1 second to 7 days, preferably 2-24 hours, more preferably 4-6 hours; followed by
(c3) optionally increasing the temperature to between about −20° C., preferably about 5-30° C., more preferably about 10-25° C., at a pressure. of about 0-1000 Pa, preferably about 10-100 Pa, morepreferably about 20-50 Pa and incubating for about 0.05 minute to 7 days, preferably for about 0.07 minute-24 hours, most preferably 0.1 minute-8 hours.

Although various methods known in the art for freeze-drying can successfully be applied (a representative run of the lyophiliser program is shown in FIG. 1), it was found that the lyophilising step as described above results in good yields of hollow particles. For example, when elastin was used, a lower pressure (e.g. 20 Pa) during lyophilisation led to the formation of more open hollow particles, whereas at a higher pressure (400 Pa) more sheet-like structures are observed. As will be understood by the person skilled in the art the effect of pressure conditions on the formation of particles according to the method of the current invention will depend on the type of solute or colloid used and can be determined by straightforward experimentation. E.g. with an increased pressure less or more sheet-like structures might be observed, whereas with a lower pressure (closer to 0 Pa) less or more open vesicles might be found.

It has surprisingly been found that by the addition of volatile organic compounds to the mixture, the formation of particles with the method according to the invention can be advantageously controlled. The term “volatile organic compounds” is known in the art and refers to organic compounds which can be essentially removed, e.g by sublimation, during lyophilisation. It has been found that the properties of such volatile compounds, e.g. the length of alkyl chains can influence (structural) properties of the hollow particles.

Therefore, according to a further embodiment of the current invention, the mixture further comprises at least one volatile organic compound, preferably capable to be essentially removed by lyophilisation.

The volatile organic compound is preferably chosen in that e.g. during the later lyophilisation step, the bulk of the volatile organic compound can easily be removed, e.g. by sublimation, e.g. from the particle wall or the lumen of the particle. The person skilled in the art can, without any inventive skill, determine, e.g. by straightforward experimentation, the suitable conditions during lyophilisation.

According to a further embodiment, the volatile organic compound comprises a carboxylic acid, preferably selected from the group consisting of formic acid, acetic acid, propionic acid and butyric acid or a combination of two or more thereof.

It has been found that for example in the case hollow particles are prepared from distinct mixtures comprising elastin (e.g 2.0% elastin in 0.25 M acetic acid, pH 3; 2.0% elastin in 0.25 M formic acid, pH 2; 2.0% elastin in 0.25 M propionic acid, pH 4) a carboxylic acid with a longer alkyl chain leads to the formation of smaller particles. This is probably due to higher propensity to phase separate from water.

It will thus be understood by person skilled in the art, that by the addition of a volatile organic compound to the mixture comprising at least one colloid or solute, e.g.. by including a carboxylic acid with a longer alkyl chain (e.g. C1-C15 or more), it is possible to adjust the diameter of the envisaged small particles, which are obtained according to the method of the current invention. By using carboxylic acids with different alkyl chain length in the mixture, particles with different characteristics.(e.g. smaller or bigger) can be formed. Preferably, carboxylic acids are chosen that can substantially be removed during lyophilisation so that the lyophilised particles are substantially free of said carboxylic acids and not present in the formed particle. The person skilled in the art can, without any inventive skill, determine, e.g. by straightforward experimentation, the suitable conditions during lyophilisation.

Preferably, the concentration of the volatile organic compounds in the mixture is 0.01-4 M, preferably 0.05-2 M, more preferably 0.1-1 M, most preferably 0.15-0.4 M. It has been shown that the use of these compounds in the above range allow for preparation of particles, and in general easy and efficient removal during lyophilisation, without leaving substantial amounts of the volatile organic compounds in the particles. The person skilled in the art can, without any inventive skill, determine, e.g. by straightforward experimentation, the suitable conditions during lyophilisation.

According to a further embodiment of the current invention the method further comprises the step (d) of stabilising the hollow particle.

Within the context of the current invention, “stabilising” refers to treating the obtained particles such that rigidity is conferred to the particles, thereby fixing e.g. the size and morphology of the particle and for example, allowing the particles to be taken up in a next medium without the particles dissolving in said next medium. As such, the particles are more resistant to e.g. decay or disintegration or unwanted or unintended modification. Suitable, methods for stabilising depend on e.g. the solute or colloid used, and are known by those skilled in the art, and may include chemical and physical cross-linking, e.g. treatment with aldehydes, radiation, heating or carbodiimides.

Preferably, stabilising is performed without negatively modifying the particle material. “Without negatively modifying” means within the context of the current invention that e.g., the properties or the structure of the particle, before stabilising, are not substantially negatively modified upon stabilising. E.g. the susceptibility towards other materials e.g. enzymes, and the properties of the particle per se, which are useful or preferred in the use of the envisaged hollow particle are not or only limited altered by the step of stabilising the particles. As will be understood by a person skilled in the art, minor loss of a property or susceptibility as mentioned above is acceptable without leaving the scope of the current invention.

If the colloid or solute comprises a glycoprotein, protein or peptide, the step of stabilising preferably comprises contacting the hollow particle with glutaraldehyde/formaldehyde vapour or glutaraldehyde solvent, or carbodiimides.

Stabilising the protein or peptide typically involves method comprised in the art, such as cross-linking (e.g. Jayakrishnan A & Jameela S R. Glutaraldehyde as a fixative in bioprostheses and drug delivery matrices. Biomaterials. 1996 March; 17(5):471-84 or Khor E. Methods for the treatment of collagenous tissues for bioprostheses. Biomaterials. 1997 January; 18(2):95-105)), and will be further detailed in the examples below.

In still a further embodiment of the current invention the colloid or solute is selected from the group consisting of protein, glycoprotein, peptide (i.e. a compound comprising less than 500 amino acids), amino acid, sugar, carbohydrate, lipoprotein, lipid, glycolipid, silica, drug, nucleic acid, DNA, RNA, vitamin, nutrient, hydrolysate, polymer, oligomer, monomer, polysaccharide, monosaccharide, recombinant peptide, bioorganic compound, recombinant biomolecules, and fragments and modifications, thereof.

The term “biomolecule” refers to any molecule or part thereof that is produced in living organisms. “Recombinant biomolecule” refers to any biomolecule or part thereof that is being biologically produced outside its natural context, for example human proteins, sugars, or parts thereof in yeast or bacterial cells, fusion-proteins and the like, e.g. obtained by genetic engineering, or by e.g. synthesis by recombinant proteins.

It is found that hollow particles with different sizes and properties can advantageously be obtained from a wide range of different colloids or solutes according to the method of the current invention. As will be understood by the person skilled in the art, any suitable molecule can successfully be applied as solute or colloid in order to form particles according to the invention. When following the current invention the skilled person in the art will, without the need for any further inventive thought, be capable of determining the suitability of the colloid or solute.

It will be understood by the person skilled in the art that one or more colloids or solutes can be combined in the mixture according to the invention in any suitable ratio. It has thus been found that the solute or colloid may be any suitable molecule with the appropriate choice of liquid medium, but the method according to the invention is advantageously applied to colloid or solutes selected from the group consisting of protein, glycoprotein, peptide (i.e. a compound comprising less than 500 amino acids), sugar, carbohydrate, lipoprotein, lipid, glycolipid, silica, drug, nuclear acid, DNA, RNA, vitamin, nutrient, hydrolysate, polymer, oligomer, monomer, polysaccharide, monosaccharide, recombinant peptide, self-assembling protein, bioorganic compound, recombinant biomolecules, and fragments and modifications thereof.

More preferably the colloid or solute is selected from the group consisting of protein, peptide, glycoprotein, carbohydrate, lipoprotein and polysaccharide. Even more preferably the colloid or solute is selected from the group consisting of protein, glycoprotein, peptide and polysaccharide. Still even more preferably the colloid or solute is chosen from the group consisting of elastin, albumin, collagen and heparin, and fragments and modifications thereof.

According to a further embodiment of the current invention the method further comprises incorporating a compound in the particle wall by adding in step the compound with the liquid medium before the freezing step. Incorporation in the particle wall was found to be achieved by adding a compound to the mixture comprising a liquid medium and at least one colloid or solute, prior to freezing said mixture, e.g. in a freezing medium.

Any suitable compound can be included in any suitable amount in the mixture.

As will be understood by the person skilled in the art, the maximal amount of the compound to be incorporated in the particle will be limited by the effect on particle formation. For example, starting from a particle obtained from a colloid or solute without the addition of a compound to be incorporated in the wall of the particle, it can be easily assessed what the maximal amount of the compound which can be incorporated in the particle wall is, by gradually increasing the amount of the compound to be incorporated in the particle wall in the mixture (e.g. in steps of 5% (w/v)). When particle formation is negatively influenced, the maximal ratio between solute or colloid and the compound to be incorporated in the particle is reached, under the given conditions or circumstances.

The compound to be incorporated in the particle material can be any suitable compound, and can advantageously be selected from the group consisting of protein, glycoprotein, peptide, sugar, carbohydrate, lipoprotein, lipid, glycolipid, silica, drug, nucleic acid, DNA, RNA, vitamin, nutrient, hydrolysate, polymer, oligomer, monomer, polysaccharide, monosaccharide, recombinant peptide, bioorganic compound, recombinant biomolecule, and fragments and modifications thereof. As will be understood by the person skilled in the art, the compound is preferably as such that it will not be removed during lyophilisation. Incorporation of said compounds provides convenient means to e.g. specifically target the particles to e.g. an organ or recognition site, or to enhance or reduce binding of the hollow particle to certain surfaces (e.g. to certain receptors) and the like (see below).

In another embodiment, the method further comprises a loading step comprising incorporating a compound in the particle lumen by incubation of the obtained hollow particle.

As will be understood, and as explained above for the incorporation of a compound in the particle wall, any suitable compound can be incorporated in the lumen of the hollow particle, preferably in amounts and ratios essentially not negatively influencing the properties of the particle.

The particles according to the invention can be used as carriers for biomolecules, drugs, DNA and other materials e.g. for targeted drug delivery in the human body. In the hollow particles, drugs can be incorporated in the lumen and/or in the particle wall. Further, different compounds can be combined is such particle, e.g. in the lumen or in the particle wall, or both.

Another intriguing application of small hollow particles is, due to their size, usage in diagnostic methods, e.g. as ultrasonic echographic imaging contrast agents to aid the visualisation of internal structures, such as the heart, liver or blood vessels. Thereto, the particles can comprise contrast agents in their lumen, but also in the particle wall.

In another embodiment, the method according to the invention, wherein the colloid or solute comprises a protein or peptide and wherein the loading step is preceded by contacting the hollow particle with glutaraldehyde/formaldehyde vapour to obtain a pre-stabilised hollow particle, the loading step is followed by contacting the loaded particle with a liquid medium comprising glutaraldehyde to obtain a stabilised loaded particle.

In another embodiment, particles can be loaded with more than one compound. It will be understood that according to the present invention, suitable compounds can be incorporated in the lumen of a hollow particle, and/or in the particle wall of a hollow particle, or throughout the particle material in case of particles with a very small lumen, or in layers thereof, or combinations thereof. A first compound can be incorporated in the wall of a hollow particle, whereas another compound can be loaded in the lumen of the same or another hollow particle. Likewise it will be understood that. different compounds can be incorporated in the wall of a particle or in the lumen of a particle. In this manner it is now possible to e.g. include an enzyme substrate in the lumen of the hollow particle, and include the enzyme in the wall of the hollow particle or include a prodrug/proenzyme in the lumen of the hollow particle and an activating compound in the wall of the hollow particle.

It will also be understood that two or more different types of particles can be combined, wherein e.g. in a first type of hollow particles, a compound is incorporated in the lumen or the wall of the particle and wherein in a further type of hollow particle another compound is incorporated in the lumen or the wall of the hollow particle. In this manner it is now possible to e.g. include a substrate for an enzyme in one type of particle, and include the enzyme or a co-factor of the enzyme or an activator of the enzyme in another type of particle obtained according to the invention.

It has thus now surprisingly been found that the method according to the invention allows for the formation of a wide range of particles. The versatile method allows the person skilled in the art by modifying any parameter discussed herein to obtain an envisaged particle. By performing the method according to the invention and observing particle formation, a person skilled in the art can, with the teaching of the current invention and without any inventive skill, by experimentation suitably adjust one or more of the parameters influencing particle formation, as discussed throughout the current invention, and subsequent observe particle formation in order to obtain a suitable particle. Thus, by step-wise adjusting parameters within the context of the current invention, and observing and comparing particle formation, it is now possible to obtain any said suitable hollow particle. Therefore, according to a further embodiment of the current invention there is provided a method for the preparation of hollow particles from at least on colloid or solute, the method comprising.
(1) providing a mixture comprising a liquid medium A and at least one colloid or solute B at a concentration C, and optionally comprising a volatile organic compound D at a concentration E;
(2) subjecting at least 0.1 μl of the mixture of step (1) to a freezing step comprising: (a) quench freezing the mixture at a temperature G and incubating said quench frozen mixture for a period H₁ at a temperature J₁, which is above the temperature G and below the melting point of the liquid medium A, or (b) reducing the temperature of the mixture at a rate of F ° C./minute to below the freezing temperature of the mixture, and incubating said frozen mixture for a period H₂ at a temperature J₂, which is above the glasstemperature of the mixture and below the melting point of the liquid medium A;
(3) lyophilising the obtained frozen mixture of step (2a) or the frozen mixture of step (2b);
(4) checking for the presence of hollow particles in the lyophilised material of step (3) and if no hollow particles or an insufficient number thereof can be observed, repeating steps (1)-(4), wherein at least one of A, B, C, D, E, F, G, H₁, H₂, J₁ or J₂, is adjusted.

The current invention enables the formation of hollow particles from a solute or colloid. As will be understood by the person skilled in the art, and without leaving the scope of the current invention, conditions of the method will in part depend on the solute or colloid. used. By varying at least one of A, B, C, D, E, F, G, H₁, H₂ J₁ or J₂, as described above, and comparing particle formation to a previous obtained result of particle formation according to the invention, the person skilled in the art will advantageously be capable of determining whether particle formation under these conditions is advantageously modified. Particular in the case no hollow particles can be observed, adjusting at least one of the parameters is essential for establishing suitable conditions. Also in case an insufficient number of hollow particles is observed (e.g. when less than 10% of the material obtained are the envisaged particles), further adjustment of the parameters and comparison allows for determining suitable conditions. By subsequently adjusting the same or any other parameter discussed herein, further modification of the particles can be observed, eventually allowing for obtaining the envisaged particles within the scope of the current invention. Once suitable parameters have been established, the method according to the invention, with the suitable parameters, can be applied for producing the particles, e.g. on industrial scale. This is further detailed in the examples below and has been discussed above.

It is to be understood that this method can also be used to obtain small particles (i.e. in the nano- and micrometer range) of any desired shape, size and volume. In such case, in step 4 it is checked for the presence of particles of the desired shape, size, and/or volume, and if no such particles or insufficient number thereof are observed, repeating step (1)-(4) wherein at least one of A, B, C, D, E, F, G, H₁, H₂, J₁ or J₂ is adjusted.

According to another embodiment of the current invention the lyophilising at step (3) above comprises the steps of (3a) applying a temperature K at a pressure L for a period M; followed by(3b) increasing the temperature to N over a period P; followed by (3c) optionally increasing the temperature to Q at a pressure R and incubating for a period S; and wherein step (4) comprises the step of checking the presence of hollow particles in the lyophilised material of step (3) and if no hollow particles or an insufficient number thereof can be observed, repeating steps (1)-(4), wherein at least one of K, L, M, N, P, Q, R, S is adjusted.

By changing one of the parameters above and observing particle formation and comparing to a previous obtained particle, the person skilled in the art will be capable, without any inventive skill, to determine whether changing said parameter has substantially improved the formation of an envisaged particle. The comparison thus allows for determining whether further adjustment of said parameter is required and/or whether adjustment on any other parameter as discussed herein is required. By repeating the method according to the invention and stepwise adjusting a parameter during each experiment, the person skilled in the art will, without any inventive skill be capable of obtaining the envisaged particle.

As described above any suitable lyophilising step can be applied within the context of the current invention. It has been found that advantageously, by adjusting on of K, L, M, N, O, P Q, R, S, as described above, the person skilled in the, art is capable (e.g. in case a globular structure/particle is obtained), to suitably adjust the lyophilising step according to the current invention, in order to obtain the envisaged particle according to the invention. By changing one of the parameters above and observing the presence of particles in the lyophilised material of step (3) above and comparing to a previous obtained particle, the person skilled in the art will be capable, without any inventive skill, to determine whether changing said parameter has substantially improved the formation of an envisaged particle. The comparison thus allows for determining whether further adjustment of said parameter is required and/or whether adjustment on any other parameter as discussed herein is required. By repeating the method according to the invention and stepwise adjusting a parameter during each experiment, the person skilled in the art will, without any inventive skill be capable of obtaining the envisaged particle.

Various methods known to the person skilled in the art can be used, e.g. electron microscopy (EM; as explained in detail in the examples below), to determine the particle nature of the structures obtained, such as the hollow nature of globular structures. The person skilled in the art will understand that the properties of the particle might depend on the colloid or solute used and the various other experimental conditions applied within the context of the current invention.
In a further preferred embodiment, the parameters within which the person skilled in the art will, within the scope of the current invention, vary is as follows:
A is selected from the group that consist of water, organic compound comprising liquid medium, volatile liquid medium, inorganic compound comprising liquid medium, acid liquid medium; and or
B is selected from the group consisting of protein, glycoprotein, peptide, sugar, carbohydrate, lipoprotein, lipid, glycolipid, silica, drug, nucleic acid, DNA, RNA, vitamin, nutrient, hydrolysate, polymer, oligomer, monomer, polysaccharide, monosaccharide, recombinant peptide, self-assembling peptide bioorganic compound, recombinant biomolecule, and fragments and/or modifications thereof; and/or
C is between 0.001-500 mg/ml (w/v) liquid medium; and/or
D is selected from the group consisting of formic acid, acetic acid, propionic acid and butyric acid or a combination of two or more thereof; and/or
E is between 0-4M; and/or
F is between 1° C. and 100° C.; and/or
G is between about −270° C. and 0° C.; and/or
H₁, H₂ is between 0.1 second-7 days; and/or
J₁, J₂ is between −200° C. and 0° C.
K is between −120° C. and 0° C.; and/or
L is between 0-1000 Pa; and/or
M is between 0,1 second-7,days; and/or
N is between −120° C. and +40° C.; and/or
P is between 0.1 second-7 days; and/or
Q is between −20° C. and +40° C.; and/or
R is between 0-1000 Pa and/or
S is between 0-7 days.

In addition to the formation of hollow particles with a well-defined lumen, it has thus been found that the method allows for the formation of particles wherein the volume of the lumen is reduced or even absent, thus providing particles wherein no lumen is present, e.g.,massive particles of any desired shape, size and volume, with dimensions in the nano- and micrometer range. Thus, there is further provided a method for the preparation of a particle having a dimension of between 1 nm and 100 μm of any required size, and shape, wherein step (4) comprises checking for particles of the said required size and shape, and if no such particles or insufficient numbers thereof can be observed, repeating steps 1-4 wherein at least one of A, B, C, D, E, F, G, H₁, H₂, J₁ or J₂ is adjusted. Any of the in the invention described steps or conditions can also suitably applied to the above method for the preparation of a particle having a dimension of between 1 nm and 100 μm of any required size, and shape, wherein step (4) comprises checking for particles of the said required size and shape, and if no such particles or insufficient numbers thereof can be observed, repeating steps 1-4 wherein at least one of A, B, C, D, E, F, G, H₁, H₂, J₁ or J₂ is adjusted. For example, the described stabilising of the particles and loading of materials in the particle material.

Within the above given ranges, particles, in particular hollow particles might be obtained with any suitable solute or colloid according to and in context of the current invention, as will be exemplified in further detail in the included examples. Based on the experimental outcome of adjusting at least one of the above given parameters, the person skilled in the art will be capable of determining further adjustment to the given parameters. For example, with low colloid or solute concentrations, e.g. elastin, tyroid-like structures can be observed. With high solute or colloid concentrations, more sheet-like structures might be formed, e.g. because separate globules might not be created. With another volatile organic compound (e.g. comprising a carboxylic acid with a longer alkyl-chain), globule size might be smaller. With a slower freezing rate, more sheets might be present. With a higher freezing rate, the presence of sheets (e.g. for elastin) might be less. A longer incubation time or a higher incubation temperature might result in larger particles, in particular larger hollow particles, and ultimately sheet-like structures, whereas a shorter incubation time and/or a lower incubation temperature might result in smaller particles, in particular smaller hollow particles.

It will be clear to the person skilled in the art that by varying the different conditions, means are provided to control hollow particle properties such as diameter, size, volume of the lumen, thickness of the wall (varying e.g. from one molecular layer thick to half the diameter of the particle), and others. For example, larger hollow particles may e.g. be prepared with the use of a carboxylic acid with a smaller alkyl chain, a slower freezing rate, a longer incubation period or a higher incubation temperature.

Likewise the role of the other parameters given in the formation of particles can easily be determined by the person skilled in the art, thus enabling the person skilled in the art in optimising, within the context of the current patent application, the method for obtaining particles, in particular hollow particles, according to the invention.

In another aspect the current invention relates to the particles, obtainable by the method as described herewith.

Another aspect of the invention relates to hollow particles wherein the wall of the particle comprises at least 80% (w/w) protein, hydrolysate of protein or a combination thereof are provided. The hollow particle provided is a globular structure and the lumen of said hollow particle can be empty or can be loaded, for example according to the method of the current invention. Next to protein, hydrolysate of protein or a combination thereof, the particle wall can further comprise any suitable compound, for example a drug, a lipid, a carbohydrate and the like.

The term “hydrolysate of protein” refers to the product of hydrolysis of a protein that comprises a mixture of amino acids and peptides. In case of a total hydrolysate the mixture of amino acids and peptides is in ratios that essentially correspond with the ratio thereof in the protein of origin. Methods for the preparation of hydrolysates of protein are known in the art and can for example involve enzymatic or acid hydrolysis. Hydrolysates of protein can be prepared from more than one protein, either in one reaction, or in separate reactions, and can for example be combined with protein or other hydrolysates of protein, but can also be a partial hydrolysate or a fraction of a (partial) hydrolysate. Likewise, different proteins can be combined to form at least 80% (w/w) of the particle wall. Thus, there is now for the first time provided hollow particles prepared from glycoproteins, proteins, hydrolysates of (glyco)protein or a combination thereof. Preferably, the hollow particles do not comprise substantial amounts of lactose, chitosan or diblock polymers. In another embodiment, particles wherein the particle wall comprises at least 80% (w/w) elastin, albumin, collagen, hydrolysate therefore, or a combination thereof are provided. In another embodiment particles wherein the particle wall comprises at least 80% (w/w) heparin are provided.

In a further aspect, the invention relates to the use of particles, in particular hollow particles, obtainable or obtained by the method of the current invention for the preparation of a medicament. As explained, a hollow particle can be provided with at least one compound like a drug, prodrug or biomolecule present in e.g. the particle wall or the lumen, or both, of the particle. The particle can successfully be designed to be applied to a patient. For example, in case of oral intake, the particle can be designed as such that it can resist the conditions in the gastrointestinal tract, by choosing a solute or colloid or stabilisation method which provides a particle that is resistant to the conditions present in the intestinal system (acidic conditions, enzymes, mechanical pressure and others). Alternatively, (hollow) particles can be designed as such that they can be activated or modified by the environments, e.g. by the acidic conditions in the stomach.

If for example, the particle according to the invention is to be applied topical, it can be designed to be easily internalised by e.g. the skin, or, if required, to not be internalised. When, for example, the particle is injected in either the bloodstream, or directly into tissue or organ, the particle can be designed to e.g. be small enough to flow through the bloodstream, or to be specifically degraded or activated at a target tissue or organ. The latter can be achieved by e.g. including a ligand or molecule binding to a ligand, e.g. an antibody, hormone, growth factor, receptor or cytokine and the like in the wall of the hollow particle, that specifically binds at the target, or by designing the particle as such that it will be degraded, e.g. by enzymes, at the target site, for example proteases, elastase, collagenase, and trypsin.

Advantageously and like above, the particle, in particular the hollow particle, obtainable or obtained by the method according to the invention can be used in a method for diagnoses of treatment of the body, tissue engineering, drug delivery, controlled release, controlled delivery, analysis, storing, protecting, targeting or isolating compounds.

In a further embodiment the particle, in particular the hollow particle, obtainable or obtained by the method according to the present invention can be used in the treatment or diagnoses of dermatological conditions, internal conditions, or cosmetics. In addition, the particles according to the invention can for example be used as a prodrug, and in veterinary, agricultural, paint, glue, military, biotechnology, chemistry, antibiotics, and coating applications, and in analytical techniques e.g. ELISA and chromatography.

According to even another aspect the current invention relates to a composition comprising a particle obtainable by the method according to the current invention wherein the composition further comprises at least one compound selected from the group consisting of a buffer, pharmaceutical acceptable carrier, a viscosity affecting compound, a tonicity affecting compound, a preservative, a cofactor, a catalyst, a substrate, an inhibitor, a nutrient, a vitamin, an enzyme, a drug, an antibody, a contrast fluid, a magnetic compound, a label, a gas, or a combination of 2 or more thereof.

Preferably the composition comprises a hollow particle according to the invention in a form selected from the group. consisting of powder, solution, capsule, liquid, dispersion, tablet, gastrointestinal tract resistant capsule, suppository, cream, foodstuff or oil.

### FIGURES

FIG. 1 shows a representative run of the lyophiliser program for the preparations of hollow elastin particles.

FIG. 2a shows a scanning electron micrograph (SEM) showing globular structures of hollow elastin particles obtained by the method according to the invention from 2.0% (w/v) solubilised elastin in medium further comprising 0.25 M acetic acid. Bar is 5 μm.

FIG. 2b shows the hollow particle nature of the globules of FIG. 2a, the even distribution of elastin throughout the hollow particle wall and the possible plasticity of the hollow particles. Bar is 1 μm.

FIG. 2c shows SEM micrographs of stabilised elastin hollow particles that were sorted based on size by using fluorescence-activated cell sorting (FACS). Bar is 2 μm.

FIG. 3 shows incorporation of probes in the hollow particle wall and hollow particle lumen. Alexa Fluor488 conjugated molecules are incorporated in the hollow particle outer layer (wall), Alexa Fluor594 conjugated molecules are present in the lumen of the same hollow particle. Bar is 2 μm.

FIG. 4 shows the effect of different parameters on the morphology of structures after freezing and lyophilisation, as analysed by scanning electron-microscopy. (a) morphology as a function of elastin concentration. (b) morphology as a function of freezing regime. Bar is 5 μm.

FIG. 5 shows particles formed by the method according to the invention from,(a) 0.25% type 1 atelocollagen, (b) 0.25% bovine serum albumin and (c) 1.0% heparin. Bar is 10 μm.

FIG. 6a shows the formation of nanoparticles in time upon enzymatic degradation of elastin hollow particles obtained by the method according to the invention, as observed by SEM. Bar is 1 μm.

FIG. 6b shows the release of fluorescent probes in time upon enzymatic degradation of elastin hollow particles obtained by the method according to the invention, as observed by confocal microscopy. It is observed that compounds are released more rapidly from the hollow particle lumen than from the hollow particle outer layer. Bar is 20 μm.

FIG. 7 shows the morphology of scaffolds as analysed by scanning electron microscopy of a non-crosslinked (NX) (A) and EDC/NHS-crosslinked (X) COL-ELsol scaffold (B) and by light microscopy using toluidine blue stained sections of EDC/NHS-crosslinked (X) COL-ELsol scaffolds (C-D). White arrows indicate hollow particles present in the scaffolds. EDC/NHS-crosslinked scaffolds contained both closed (black arrowheads) or open (black arrows) elastin particles. Bar is 10 μm in A, B and 20 μm in C, D.

### EXAMPLES

### Example 1

Preparation of elastin.

Purified insoluble elastin fibres were prepared as described (Daamen W F, et al. Tissue Eng., 2005; 11:1168-1176) and hydrolysed with a procedure based on the method described by Partridge. (Partridge S M et al. Biochem J. 1995; 61:11-21).

Generally, elastin was solubilised after. 14 1-hour hydrolysis steps with 0.25 M oxalic acid at 100° C. Supernatants were pooled, and dialysed against 10 mM phosphate buffer pH7.4 and then against MilliQ water. The solubilised elastin preparation (referred to as “elastin”.) had a mean molecular mass of about 1100 kilodalton (kDa) with a large molecular mass distribution.

### Example 2

Preparation of particles from elastin.

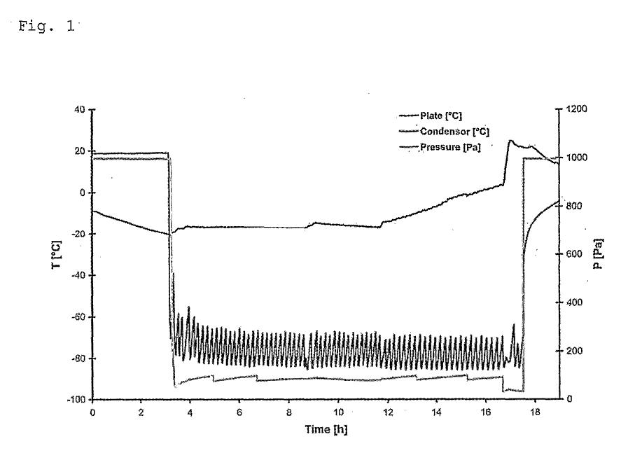

Droplets of about 20 μl 2.0% (w/v) elastin in 0.25 M acetic acid were immersed in liquid nitrogen for about one minute. The frozen droplets were then incubated at
−10 to −20° C. for about 3 hours. Subsequently the sample was lyophilised in a Zirbus lyophiliser. (Sublimator 500 II Bad Grund, Germany) using the program plotted in FIG. 1. For this, a temperature of about −20° C., which is above the freezing temperature of the mixture, at a pressure of about 50-200 Pa, was applied for a period. of about 12 hours, followed by increasing the temperature to about 0° C. over a period of about 4 hours; followed by increasing the temperature to about 20° C., at a pressure of about 30 Pa and incubating for about 1 hour.

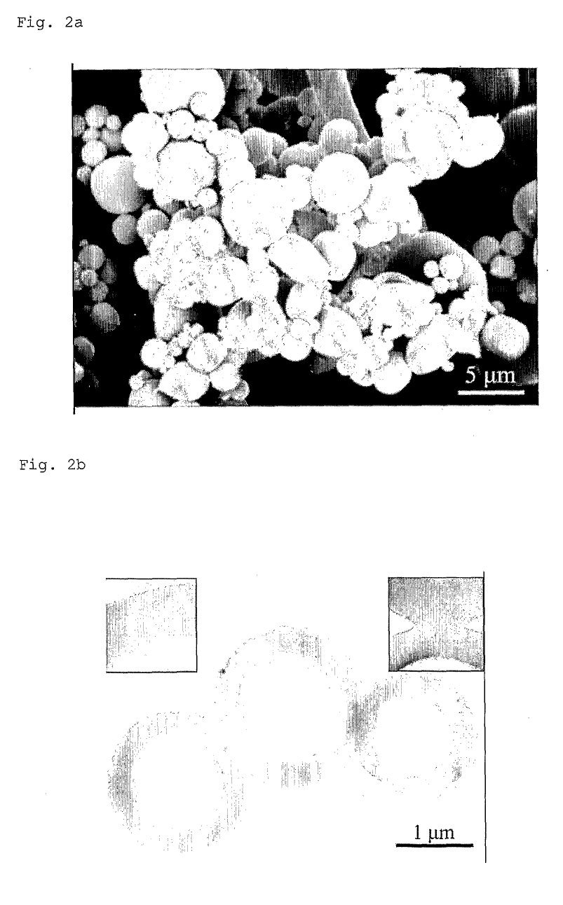

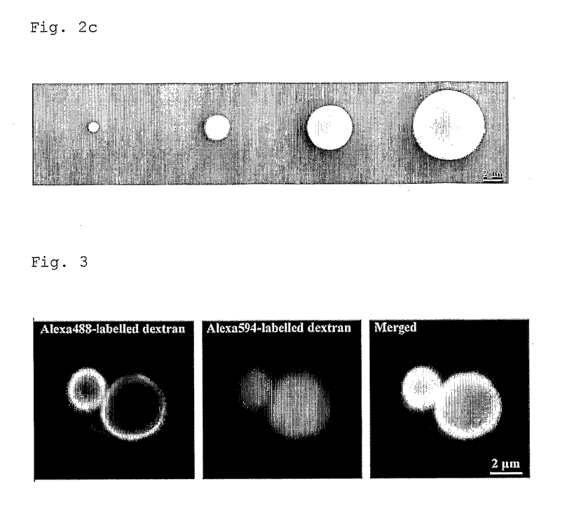

By following the above-mentioned procedure globular structures were formed ranging from 0.25-1.0 micrometer in. diameter as revealed by scanning electron microscopy (SEM) (FIG. 2a). For SEM, the lyophilised samples were sputtered with gold and studied with a JEOL JSM-6310 SEM apparatus (JEOL, Tokyo, Japan; according to manufacturer's instructions) with an accelerated voltage of 15 kV. Wet samples were first critical point dried using CO2 (Lieu et al. J Control Release 2002; 78:259-266). Further analyses using transmission electron microscopy (TEM) showed the hollow particle nature of these globular structures (FIG. 2b). Elastin hollow particles with a perfect smooth and round morphology were obtained and elastin is equally distributed throughout the hollow particle wall.

For transmission electron microscopy (TEM) the samples were post-fixed with 1% w/w osmium tetroxide in 0.1 molar phosphate buffer (PB) for 1 H, optionally after vapour and wet stabilisation (see below). After a rinsing period of 3 hours with 0.1 molar PB, samples were dehydrated in an ascending series of ethanol in water solutions 30%, 50%, 70%, 90%, 100% ethanol), embedded in epoxy resin (EPON 812), and microtomed (see Meek J. et al. J Comp Neurol. 2001 Mar 12; 431(3):255-75). Ultra-thin sections (60 nanometer) were picked up on formvar-coated grids, poststained with lead citrate and uranyl acetate and examined in a JEOL 1010 electron microscope (JEOL, Tokyo, Japan). Alternatively, hollow particles were obtained when the. mixture comprising elastin was frozen by reducing the temperature of the mixture at a rate of about −30° C./minute. For this, 10 ml of the mixture was poured into a plastic mould, frozen in a bath of ethanol and solid CO2 (−80° C.) and lyophilised in a Zirbus lyophiliser (Bad Grund, Germany), using the same conditions as above. Optionally, the said frozen mixture can be incubated at a temperature above −120° C. for a period of e.g. 4 hours, before lyophilisation. Hollow particles were obtained, as was observed by TEM as described above.

### Example 3

Stabilisation of hollow particles.

After preparation the elastin hollow particles were stabilised by treatment with a glutaraldehyde/formaldehyde vapour during a period of 48 hours (“Vapour fixation”). For this, the particles were placed in a container in which a 25% glutaraldehyde/38% formaldehyde 1:1 aqueous solution was placed.

Optionally, further stabilisation can be performed by cross-linking in a solution of 0.5% glutaraldehyde in phosphate buffer of pH 7.4 for a period of 4 hours “wet fixation”, further increasing rigidity of the obtained particle, and, as discussed below, trapping compounds incorporated in the particle, e.g., lumen in a hollow particle.

### Example 4

Analysis and sorting of hollow particles by fluorescence-activated cell sorting (FACS).

Using a flow cytometer (Epics Elite flow cytometer, Coulter, Luton, UK) hollow particles could be sorted according to size (by normal procedures including forward and side scatter), and it was determined that in the case of elastin the diameter of stabilised vesicles was up to 10 micrometer as shown in FIG. 2c, as studied by SEM (as above, see example 2).

### Example 5

Incorporation of compounds (fluorescent) into hollow particles.

To a mixture comprising a liquid medium and 2.0% w/v elastin, 50 microgram probe per ml was added prior to immersing a droplet of the mixture in liquid nitrogen. Fluorescent probes included Alexa Fluor 594 labelled goat anti-mouse antibody and Alexa Fluor 488/594 labelled-Dextran (10000 Dalton).

Incorporation of fluorescent probes in the hollow core of the particle (lumen) was performed by a 96 hours incubation of vapour-fixed particles (see above) in a solution of 50 microgram probe/ml in. either MilliQ (dextrans) or ethanol (DiOC₁₈; see below), followed by wet fixation, and 3 times washings with milliQ or 100% ethanol to remove non-included probe. Probes included Alexa Fluor 488 or 594-labelled Dextran (10000 Dalton) and 3,3′ dioctadecyloxacarbocyanine perchlorate DiOC₁₈; all from molecular probes Europe (Leiden, the Netherlands).

The presence of the probes in the hollow particle was studied using confocal microscopy. For this hollow particles with incorporated probes were deposited on poly-d-lysine coated coverslips and confocal images were made at 488 nanometer and 594 nanometer with a Biorad MRC1024 confocal laser scanning microscope, equipped with an argon/crypton laser, using a 60×1.4 NA oil objective and LaserSharp 2000 acquisition software.

Results show that fluorescent probes were present in the hollow particles in either the hollow particle wall and/or in the hollow particle lumen/hollow core, depending on the applied techniques described above (FIG. 3).

It is clear from this example that the hollow particles according to the invention are suitable for differentially incorporating similar substances into the hollow particle wall and/or lumen or to incorporate two distinct substances in the hollow particle wall and/or lumen. E.g. (fluorescent labelled) antibodies in the wall and (fluorescent labelled) dextrans in the lumen, or a hydrophilic probe in the wall, and a lipophilic (e.g. DiOC₁₈) probe in the lumen of a hollow particle. It will be understood by the person skilled in the art that the possibilities are not limited to the examples given above.

### Example 6

Parameters influencing vesicle formation.

The methodology described in Example 2 involved 2% (w/v) elastin (w/v) in 0.25 molar acetic acid liquid medium of which about. 20 μl was immersed in liquid nitrogen to form frozen droplets. The sample is subsequently placed in a lyophiliser with a plate temperature of −10° C. that gradually decreased to −20° C. within 3 hours. When the plate temperature reaches −20° C pressure was reduced (80 Pa) and these settings were kept constant for approximately 8 hours. The plate temperature was the increased to 0° C. over a period of approximately 5 hours. Next, the plate temperature was increased to 20° C. and pressure decreased to 30 Pa and kept for approximately 1 hour. Finally, the lyophiliser was slowly aerated and the samples were taken out of the lyophiliser.

To study the influence of various parameters on particle formation, parameters, including but riot limited to concentration of the colloid or solute, medium composition, freezing temperature and rate, incubation conditions, pressure and temperature conditions during lyophilisation and type of colloid or solute used, were varied.

Modification in any of these parameters results in altered morphology of the structures obtained as can be witnessed from below.

As will be understood by a person skilled in the art the ranges and effect of the variations depend on the type of solute or colloid used. Although the observed influence of parameter variation can be considered to describe a general principle, it will be understood that the relative contribution of the different parameters will depend on the e.g. solute or colloid used. It will also be understood that the experiments below are applicable for all types of particles, including solid and hollow particles according to the invention.

1. Colloid or solute concentration.

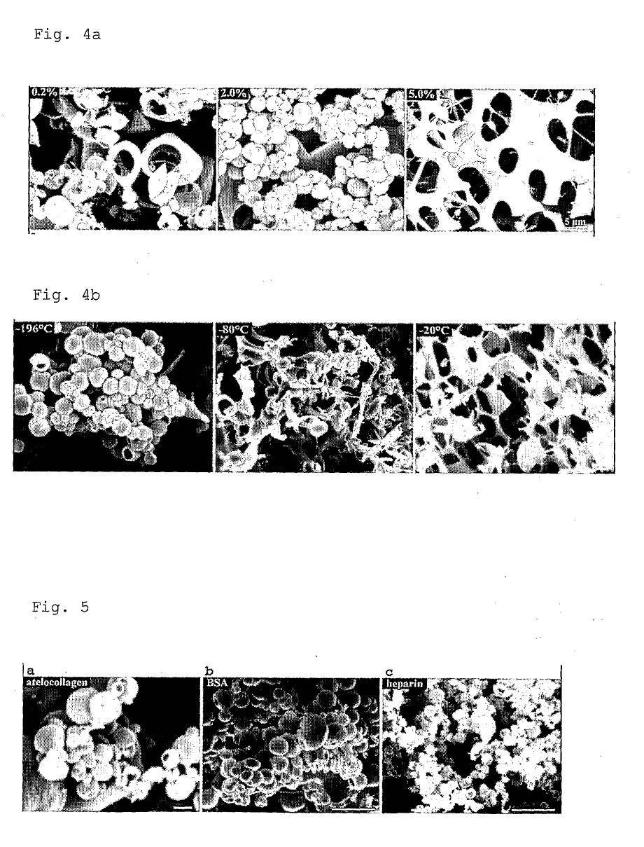

It was found that when concentration of the colloid or solute was varied, the type of structures obtained formed after freezing and lyophilisation varied. At an elastin concentration of 2.0% (w/v) mostly hollow particles were obtained. When lower concentrations were used (0.2% (w/v)) other self-assembled structures were found, including tyroid-like structures and open structures. At higher concentrations (5.0% (w/v)) hollow and solid sheets were predominately found. At a concentration of 2.0% (w/v), the majority of the structures in the preparations were hollow globules (FIG. 4a).

2. Medium composition.

Medium composition was varied to study the effect on the hollow particles obtained. When 0.25 M formic acid, acetic acid, propionic acid or butyric acid was comprised in the medium, more globular structures were formed (e.g 0.25 M acetic acid, pH=2.5). Globule size was smaller with increasing alkyl chains of the acid solvent. Globules turned out to be hollow particles as analysed with TEM (see above).

3. Temperature of the freezing medium (freezing rate).

Variation in the temperature of the freezing medium, and thereby in the freezing rate lead to variation in the type of particle obtained. Freezing 2.0. % (w/v) elastin in medium further comprising 0.25 M acetic acid in liquid N2 and subsequent lyophilisation yields hollow particles. However, when the freezing rate is slowed down by using a solid CO2 ethanol mixture (−80° C.) or by placing the sample in a −20° C. freezer, more sheet-like structures, were also found (FIG. 4b).

4. Incubation regime.

With the procedure as explained in examples above, frozen samples were incubated in a −10 to −20° C. environment for 3 hours. When this time period is prolonged, more sheet-like structures (instead of discrete particles) were found after lyophilisation. At lower temperatures (e.g. −80° C.) the hollow particles obtained from elastin after lyophilisation were observed to be smaller. Freezing and/or incubating per se is required to obtain globules as is shown by microscopic analyses of frozen elastin preparations. After freezing and incubating, but before lyophilisation, globular structures were found that could be attributed to the solid or colloid used, e.g. elastin. With the use of TEM, it is observed that thread-like structures with globular extensions were found when the medium is frozen in liquid N2 and freeze substituted in acetone. Using light microscopy, elastin globules and particles (1-2 micrometer) were found when a mixture comprising elastin and a liquid medium was frozen at −20° C. per minute until −70° C. Some of the globules were attached to a thread-like network. Globules formed out of the thread-like structures when the temperature was increased, as was shown by fluorescence microscopy or from liquid nitrogen frozen samples that were left to thaw. When elastin preparation was completely melted (e.g. above the melting temperature of the liquid medium and above the freezing temperature of the mixture comprising the liquid medium and the solute or colloid, no globular structures could be observed.

5. Pressure conditions during lyophilisation

As witnessed in the case of hollow particles prepared from elastin, pressure settings during lyophilisation influenced hollow particle formation. With the standard lyophilisation pressure (80 Pa) for elastin, many hollow particles were observed to be present. When pressure is decreased to 20 Pa, more open structures are observed, whereas at higher pressure (400 Pa) more sheet-like structures are observed.

6. Type of colloid or solute.

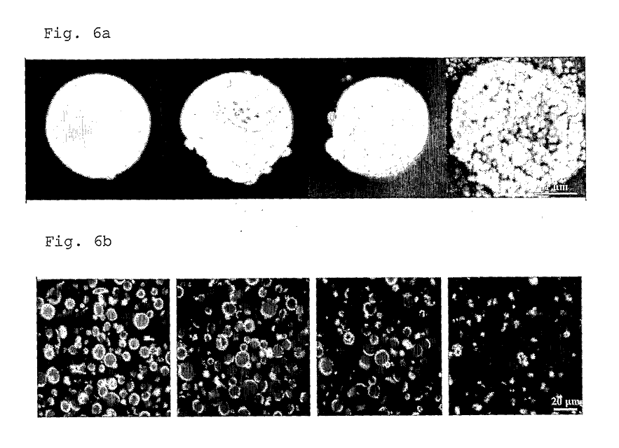

Particles were prepared from Type I atelocollagen (Symatese, Chaponost, France), Bovine albumin fraction V (Sigma, St. Louis, Mo., USA) and heparin sodium salt (from porcine intestinal mucosa; Sigma, St Louis, Mo., USA) as described for example 2, but with varying concentrations. FIG. 5 shows particles formed from (a) 0.25% type 1 atelocollagen, (b) 0.25% bovine serum albumin and (c) 1.0% heparin.

### Example 7

Particle degradation.

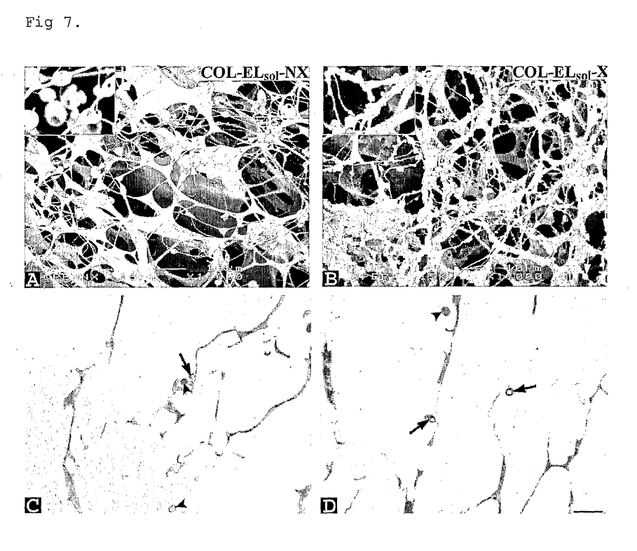

Hollow particles according to example 5, wherein fluorescent. probes were incorporated, were treated with 0.3 μl-0.4 μl per ml elastase (Sigma) in 100 mM Tris-HCl pH 8.0 analysed after 0, 15, 20 and 30 minutes at 22° C. with confocal microscopy and scanning electron microscopy as above. Degradation of the hollow particle resulted in formation of elastin nanospheres and release of the incorporated probes from the particle as shown by confocal laser scanning microscopy (FIG. 6a and 6b). This shows that an original property of the colloid or solute used, in this example elastin, can be conserved during formation of the hollow particles in accordance with the method of the current invention, even after stabilisation.

### Example 8

Use of particles in tissue engineering

Scaffolds comprising 50% insoluble type I collagen and 50% soluble elastin were prepared. For this, a 1.6%. (w/v) collagen suspension was shaken overnight in 0.5 M acetic acid at 4° C. Soluble elastin was added and the suspension was diluted with cold MilliQ water to contain 0.8% (w/v) collagen and 0.8% (w/v) elastin and subsequently homogenised on ice using a Potter-Elvehjem homogeniser. Air-bubbles were removed by centrifuging at 250 g for 10 min at 4° C. The mixture was then slowly poured into a plastic mould (10 ml mixture/25 cm2 mold; total 10 ml), frozen in a bath of ethanol and solid CO2 (−80° C.) within about 4 minutes and lyophilised (as above, in example 2) in a Zirbus lyophiliser (Bad Grund, Germany). Scaffolds were applied as such (non-crosslinked), or crosslinked. For crosslinking, 200 mg scaffold was incubated for 4 h at 22° C. with 20 ml 33 MM 1-ethyl-3-(3-dimethyl aminopropyl)carbodiimide (EDC) and 6 mM N-hydroxysuccinimide (NHS) in 50 mM 2-morpholinoethane sulphonic acid (MES) pH 5.0 containing 40% ethanol. EDC/NHS-crosslinked scaffolds were then washed with 0.1 M sodium hydrogen-phosphate (twice for 1 h), 1 M NaCl (twice for 2 h), 2 M NaCl (once overnight, 5 times 30 min) and MilliQ water (6 times 30 min). The scaffolds were then frozen in ethanol/CO2 again and lyophilised.

Under these conditions, and in accordance with the invention, it was found that hollow elastin particles were formed in the collagenous scaffolds. FIG. 7 shows the presence of such hollow elastin particles in the scaffolds prepared for tissue engineering. These composite scaffolds, including the hollow particles can be stabilised by crosslinking with the general protein-material stabilisers EDC (1-ethyl-3-(3-dimethyl aminopropyl)carbodiimide) and NHS (N-hydroxysuccinimide) (FIG. 7B-D). Different types of particles, e.g. solid particles, can be present in these type of scaffolds, but it was now surprisingly found that in particular hollow particles, in particular obtained by the method according to the current invention, can be advantageously used in tissue engineering. The particles (in this example solubilised elastin particles) appear stabile, and were found to be present in EDC/NHS-crosslinked COL-ELsol scaffolds even 21 days after subcutaneous implantation in 3 weeks old Sprague Dawley rats. It is therefore now for the first time shown that hollow particles can be advantageously used in tissue engineering. In particular particles derived from natural occurring compounds, e.g. those that naturally occur is tissue, can now be advantageously applied. Different compounds may be incorporated in these scaffolds for example, to establish a (controlled) release system. The hollow particles can for example, be loaded with various materials that are beneficial in tissue engineering, for example cytokines, drug, produgs, and the like, for example to restore tissue growth or improve acceptance of new tissue by a patient. Further, it is to be contemplated that also particles, in particular hollow particles prepared from not only natural occurring biocompounds or hydrolysates thereof, such as elastin and elastin hydrolysates, but also suitable protein fragments, or peptides as e.g. described in Bellomo et al., supra, can be used for tissue engineering. In addition to the above used freezing step of “slow” free zing the mixture, also quench freezing in accordance to the method of the invention, e.g. by freezing droplets of the mixture (20 μl) by immersion in liquid nitrogen, can be performed to provide hollow particles (e.g. elastin) within the scaffolds (data not shown).

As can be concluded from the above description and given examples, within the known variations of the method according to the current invention, particles, in particular hollow particles, with a diameter in the range of about 1 nanometer to 100 micrometer can be obtained. Parameters influencing the formation of particles can easily be varied within the method of the current invention in order to obtain hollow particles.

As different conditions influence particle formation, there is now provided a particularly interesting means to control particle parameters such as diameter and others. The hollow particles according to the invention can be used to encapsulate or enclose solutions or proteins/(pro)drugs and other suitable substances. For example, enzymes can be present in the particle wall whereas a substrate is present in the lumen of the particle, thus allowing conversion of the substrate in the particle wall, or prodrugs are present in the lumen, which, after conversion in the particle wall become active as drugs. This also applies for e.g. pro-enzymes and other precursors that can be converted to enzymes and the like. It might thus be possible to include a substrate in the lumen which can e.g. by conversion in the particle wall weaken or strengthen the particle, and thus e.g. allow for diffusion of drugs from the lumen. It might thus now also be possible to include DNA and/or other (modified) nucleic acids in the particle wall or lumen and fuse the particle with a cell, allowing for the introduction of the DNA and/or other (modified) nucleic acids in the cell.

In a pharmacological context, the simultaneous release of different materials is not easy and the preparation of multi-component particles in a single delivery vehicle is beneficial in this respect. With the provision of the possibility to incorporate different substances into e.g. the hollow particle wall and the hollow particle lumen, such a two-way system can be prepared with various colloids and solutes. Release of substance from the hollow particle can for example be tailored by the extent of stabilisation of the hollow particle, thickness of the wall and the concentration of the substances to be incorporated.

Hollow particles from e.g. naturally occurring proteins (biological proteins) are of particular interest since these are biodegradable and biocompatible. They can be used to form slow-release depots for therapeutics, may be directed to specific locations in the body (e.g. by incorporating specific antibodies into the particle wall) and may release content at specific sides (e.g. in case of elastin vesicles at the site of high elastase concentrations).

Since the hollow particles can now be prepared in large quantities application in tissue engineering is also possible.

1. Method for the preparation of hollow particles having a particle wall and a particle lumen, the particle having dimensions of between 1 nm and 100 μm, from a mixture comprising a liquid medium comprising at least one colloid or solute, the method comprising freezing said mixture and lyophilising the obtained frozen mixture, characterised in that a volume of at least 0.1 μl of the mixture is subjected to a freezing step comprising:
(a)(1) quench freezing the mixture resulting in a quench frozen mixture, and (2) incubating said quench frozen mixture at a temperature above the quench freezing temperature and below the melting point of the liquid medium, or
(b)(1) reducing the temperature of the mixture at a rate of 1 to 100° C./minute to below the freezing temperature of the mixture, and (2) incubating said frozen mixture at a temperature above the glass temperature of the mixture and below the melting point temperature of the liquid medium.

2. Method according to claim 1, wherein the volume of the mixture of step (a) is between 0. 1 μl and 1000 μl, preferably between 1 μl and 100 μl, more preferably between 2 μl and 50 μl, even more preferably between 3 μl and 30 μl, most preferably between 5 μl and 25 μl.

3. Method according to claim 1, wherein the quench freezing step (a1) comprises freezing the mixture by contacting with a freezing medium, the freezing medium having a temperature of below the freezing temperature of the mixture.

4. Method according to claim 3, wherein the freezing medium has a temperature of between −270° C. and +20° C., preferably between −230° C. and −50° C.

5. Method according to claim 3, wherein the freezing medium comprises liquid nitrogen.

6. Method according to claim 1, wherein the incubation of the quench frozen droplet is carried out at a temperature between −150° C. and 0° C., preferably between −140° C. and −0° C., most preferably between −20° C. and 0° C.

7. Method according to claim 1, wherein the volume of the mixture of step (b) is between 0. 1 ml and 100 ml, preferably between 0.5 ml and 50 ml, most preferably between 1.0 ml and 10 ml.

8. Method according to claim 1, wherein the lyophilising step comprises the steps of
(c1) applying a temperature which is below the freezing temperature of the liquid medium, at a pressure between 0-1000 Pa, preferably 20-500 Pa, most preferably 50-200 Pa, for 1 second to 7 days, more preferably 2-24 hours, most preferably 4-18 hours; followed by
(c2) increasing the temperature to between −120° C. and +40° C. over a period of 1 second to 7 days, preferably 2-24 hours, more preferably 4-6 hours; followed by
(c3) optionally increasing the temperature to between about −20° C.-+40° C., preferably about 5-30° C., more preferably about 10-25° C., at a pressure of about 0-1000 Pa, preferably about 10-100 Pa, more preferably about 20-50 Pa and incubating for about 0.05 minute to 7 days, preferably for about 0.07 minute-24 hours, most preferably 0. 1 minute-8 hours.

9. Method according to claim 1, wherein the mixture further comprises a least one volatile organic compound, preferably capable to be removed by lyophilisation.

10. Method according to claim 9, wherein the volatile organic compound comprises a carboxylic acid, preferably selected from the group consisting of formic acid, acetic acid, propionic acid and butyric acid or a combination of two or more thereof.

11. Method according to claim 9, wherein the concentration of the volatile organic compound in the mixture is 0.01-4 M, preferably 0.05-2 M, more preferably 0.1-1 M, most preferably 0.15-0.4 M.

12. Method according to claim 1, wherein the method further comprises the step (d) of stabilising the hollow particle.

13. Method according to claim 12, wherein the colloid or solute comprises a glycoprotein, protein or peptide and wherein the step of stabilising comprises contacting the hollow particle with glutaraldehyde/formaldehyde vapour, glutaraldehyde solvent or carbodiimides.

14. Method according to claim 1, wherein the colloid or solute is selected from the group consisting of protein, glycoprotein, peptide, amino acid, sugar, carbohydrate, lipoprotein, lipid, glycolipid, silica, drug, nucleic acid, DNA, RNA, vitamin, nutrient, hydrolysate, polymer, oligomer, monomer, polysaccharide, monosaccharide, recombinant peptide, bioorganic compound, recombinant biomolecule, fragments and modifications thereof.

15. Method according to claim 14, wherein the colloid or solute is selected from the group consisting of protein, peptide, glycoprotein, carbohydrate, lipoprotein and polysaccharide.

16. Method according to claim 14, wherein the colloid or solute is selected from the group consisting of protein, glycoprotein, peptide and polysaccharide.

17. Method according to claim 14, wherein the colloid or solute is chosen from the group consisting of elastin, albumin, collagen, heparin, and fragments and modifications thereof.

18. Method according to claim 1, wherein the method further comprises incorporating a compound in the particle wall by adding the compound with the mixture before the freezing step.

19. Method according to claim 1, wherein the method further comprises a loading step comprising incorporating a compound in the particle lumen by incubation of the hollow particle obtained in a liquid medium comprising the compound to be incorporated to obtain a loaded particle.

20. Method according to claim 19, wherein the colloid or solute comprises a protein or peptide and wherein the loading step is preceded by contacting the hollow particle with glutaraldehyde/formaldehyde to obtain a pre-stabilised hollow particle, and the loading step is followed by contacting the loaded particle with a liquid medium comprising glutaraldehyde to obtain a stabilised loaded particle.

21. Method according to claim 1 for the preparation of a hollow particle from at least one colloid or solute, the method comprising
(1) providing a mixture comprising a liquid medium A and at least one colloid or solute B at a concentration C, and optionally comprising a volatile organic compound D at a concentration E;
(2) subjecting at least 0.1 μl of the mixture of step (1) to a freezing step comprising: (a) quench freezing the mixture at a temperature G and incubating said quench frozen mixture for a period H₁ at a temperature J₁, which is above the temperature G and below the melting point of the liquid medium A, or (b) reducing the temperature of the mixture at a rate of F ° C./minute to below the freezing temperature of the mixture, and incubating said frozen mixture for a period H₂ at a temperature J₂, which is above the glasstemperature of the mixture and below the melting point of the liquid medium A;
(3) lyophilising the obtained frozen droplets of step (2a) or the frozen mixture of step (2b);
(4) checking for the presence of hollow particles in the lyophilised material of step (3) and if no hollow particles or insufficient numbers thereof can be observed, repeating steps (1)-(4), wherein at least one of A, B, C, D, E, F, G, H₁, H₂, J₁ or J₂ is adjusted.

22. Method according to claim 21, wherein the lyophilising at step
(3) comprises the steps of
(3a) applying a temperature K at a pressure L for a period M; followed by
(3b) increasing the temperature to N over a period P; followed by (3c) optionally increasing the temperature to Q at a pressure R and incubating for a period S; and wherein step (4) comprises the step of checking the presence of hollow particles in the lyophilised material of step (3) and if no hollow particles can be observed, repeating steps (1)-(4), wherein at least one of K, L, M, N, P, Q, R, S is adjusted.

23. Method according to claim 21, wherein
A is selected from the group that consisting of water, organic compound comprising liquid medium, volatile liquid medium, inorganic compound comprising liquid medium, acid liquid medium; and/or
B is selected from the group consisting of protein, glycoprotein, peptide, sugar, carbohydrate, lipoprotein, lipid, glycolipid, silica, drug, nucleic acid, DNA, RNA, vitamin, nutrient, hydrolysate, polymer, oligomer, monomer, polysaccharide, monosaccharide, recombinant peptide, bioorganic compound, recombinant biomolecule, self-assembling peptide and fragments and/or modifications thereof, and/or
C is between 0.001-500 mg/ml (w/v) liquid medium; and/or
D is selected from the group consisting of formic acid, acetic acid, propionic acid and butyric acid or a combination of two or more thereof, and/or
E is between 0-4 M; and/or
F is between 1° C. and 100° C.; and/or
G is between about −270° C. and 0° C.; and/or
H₁, H₂ is between 0.1 second-7 days; and/or
J₁, J₂ is between −200° C. and 0° C.

24. Method according to the claim 22, wherein
K is between −120° C. and 0° C.; and/or
L is between 0-1000 Pa; and/or
M is between 1 second-7 days; and/or
N is between −120° C. and +40° C.; and/or
P is between 1 second-7 days; and/or
Q is between −20° C. and +40° C.; and/or
R is between 0-1000 Pa and/or
S is between 0-7 days.

25. Method according to claim 21 for the preparation of a particle having dimensions of between 1 nm and 100 μm of any required size, shape, and volume wherein step (4) comprises checking for particles of the said required size, shape and volume, and if no such particles or insufficient numbers thereof can be observed, repeating steps 1-4 wherein at least one of A, B, C, D, E, F, G, H₁, H₂, J₁ or J₂ is adjusted.

26. Particles obtained by the method according claim 1.

27. Particles according to claim 26 wherein the particle wall comprises at least 80% (w/w) glycoprotein, protein, hydrolysate of protein, or a combination thereof

28. Particles according to claim 27 wherein the particle wall comprises at least 80% (w/w) elastin, albumin, collagen, hydrolysate thereof, or a combination thereof.

29. Particles according to claim 26 wherein the particle wall comprises at least 80% (w/w) heparin.

30-32. (canceled)

33. Composition comprising a particle obtainable by the method according to claim 1 wherein the composition further comprises at least one compound selected from the group consisting of a buffer, a pharmaceutical acceptable carrier, a viscosity affecting compound, a tonicity affecting compound, a preservative, a cofactor, a catalyst, a substrate, an inhibitor, a nutrient, a vitamin, an enzyme, a drug, an antibody, a contrast fluid, a magnetic compound, a label, a gas, or a combination of 2 or more thereof.

34. A composition comprising a particle according to claim 26, wherein the composition is in a form selected from the group consisting of powder, solution, capsule, liquid, dispersion, tablet, gastrointestinal tract resistant capsule, suppository, cream, foodstuff, or oil.

35. A method for diagnosis or treatment of the body, tissue engineering, drug delivery, controlled release, controlled delivery, analysis, storing, protecting, targeting or isolating, comprising administering the particle of claim 26 to a subject in need thereof.

36. A method for treatment or diagnosis of dermatological conditions, internal conditions, or cosmetics, comprising administering the particle of claim 26 to a subject in need thereof.
